(19) United States
(12) Patent Application Publication
(10) Pub. No.: US 2017/0021019 A1
(43) Pub. Date: Jan. 26, 2017

(54) COMBINATION OF IMMUNOMODULATORY AGENT WITH PD-1 or PD-L1 CHECKPOINT INHIBITORS IN THE TREATMENT OF CANCER

(71) Applicants: Matthew Zibelman, Philadelphia, PA (US); Elizabeth Plimack, Philadelphia, PA (US); Amy Grahn, Lake Bluff, IL (US); John Gerard Devane, Roscommon (IE); Jeffrey W. Sherman, Lincolnshire, IL (US)

(72) Inventors: Matthew Zibelman, Philadelphia, PA (US); Elizabeth Plimack, Philadelphia, PA (US); Amy Grahn, Lake Bluff, IL (US); John Gerard Devane, Roscommon (IE); Jeffrey W. Sherman, Lincolnshire, IL (US)

(21) Appl. No.: 15/216,585

(22) Filed: Jul. 21, 2016

Related U.S. Application Data
(60) Provisional application No. 62/195,461, filed on Jul. 22, 2015.

(51) Int. Cl. A61K 39/395; C07K 16/28; A61K 38/21

(52) U.S. Cl. CPC A61K 39/39558; A61K 38/217; C07K 16/2818; C07K 16/2827; A61K 2039/507

(57) ABSTRACT

Methods for treating cancer include administering a therapeutically effective amount of an immunomodulatory agent such as interferon gamma 1b in combination with a therapeutically effective amount of a PD-1 checkpoint inhibitor such as nivolumab or pembrolizumab or a therapeutically effective amount of a PD-L1 checkpoint inhibitor.

This application claims the benefit of priority to U.S. Provisional Application No. 62/195,461, filed Jul. 22, 2015, which is incorporated by reference herein for all purposes.

Pharmacologic approaches for treating cancer have traditionally relied on the use of various single agent systemic therapies (monotherapies). An archetypical example is chemotherapy, which utilizes broadly cytotoxic drugs that target rapidly dividing cells, including alkylating agents like dacarbazine (DTIC) or temozolomide (TMZ), or mitotic inhibitors like paclitaxel, to inhibit or kill the rapidly growing cells typical of cancer.

Cancerous tumors may not be completely responsive to such monotherapy, either due to their high collateral systemic toxicity necessitating lower, even sub-therapeutic doses or development of tumor resistance that circumvents the activity of the monotherapy agent.

Combination of systemic immunomodulatory agents with PD-1/PD-L1 checkpoint inhibitors has been proposed as means to attack cancer simultaneously via several different paths, thereby increasing potency while reducing likelihood of resistance.

One such immunomodulatory agent, interferon-gamma Type II interferon and which is commercially available as interferon gamma 1b which has been previously studied in a variety of solid tumors, but is currently only approved for the treatment of chronic granulomatous disease (CDG) and severe, malignant osteopetrosis (SMO). This Type II interferon has been shown to be a key regulator of PD-L1 expression.

Accordingly, additional advancements are needed in the fields of combination immunotherapy with IFN-γ 1b and PD-1 pathway inhibition which are proven to be safe, well tolerated, and increase the overall response rate (ORR) as compared to PD-1 pathway inhibitors alone in cancer patients with relapsed/refractory metastatic disease.

Provided is a method for treating cancer, comprising administering to a patient in need thereof a therapeutically effective amount of an immunomodulatory agent and a therapeutically effective amount of a PD-1 checkpoint inhibitor and/or a therapeutically effective amount of a PD-L1 checkpoint inhibitor.

Also provided is a method for treating cancer, comprising administering to a patient in need thereof a therapeutically effective amount of an immunomodulatory agent and a therapeutically effective amount of a PD-1 checkpoint inhibitor.

Also provided is a method for treating cancer, comprising administering to a patient in need thereof a therapeutically effective amount of an immunomodulatory agent and a therapeutically effective amount of a PD-L1 checkpoint inhibitor.

Also provided is a pharmaceutical composition, comprising: a therapeutically effective amount of an immunomodulatory agent; and a therapeutically effective amount of a PD-1 checkpoint inhibitor.

Also provided is a pharmaceutical composition, comprising: a therapeutically effective amount of an immunomodulatory agent; and a therapeutically effective amount of a PD-L1 checkpoint inhibitor.

Also provided is a pharmaceutical composition, comprising: a therapeutically effective amount of an immunomodulatory agent; a therapeutically effective amount of a PD-L1 checkpoint inhibitor; and a therapeutically effective amount of a PD-1 checkpoint inhibitor.

### BRIEF DESCRIPTION OF THE DRAWINGS

FIG. 1 shows a study schematic for interferon-gamma 1b (IFN-γ 1b) and PD-1/PD-L1 checkpoint inhibitor treatment plan.

Provided is a method for treating cancer, comprising administering to a patient in need thereof a therapeutically effective amount of an immunomodulatory agent and a therapeutically effective amount of a PD-1 checkpoint inhibitor and/or a therapeutically effective amount of a PD-L1 checkpoint inhibitor.

Also provided is a method for treating cancer, comprising administering to a patient in need thereof a therapeutically effective amount of an immunomodulatory agent and a therapeutically effective amount of a PD-1 checkpoint inhibitor.

In some embodiments, the PD-1 checkpoint inhibitor is administered substantially at the same time as the immunomodulatory agent. In some embodiments, the PD-1 checkpoint inhibitor is administered prior to administration of the immunomodulatory agent. In some embodiments, PD-1 checkpoint inhibitor is administered after administration of the immunomodulatory agent to the patient.

PD-1 checkpoint inhibitors include but are not limited to molecules that decrease, block, inhibit, abrogate or interfere with signal transduction resulting from the interaction of PD-1 with one or more of its binding partners, such as PD-L1 and PD-L2. In some embodiments, the PD-1 checkpoint inhibitor inhibits the binding of PD-1 to its binding partners. In some embodiments, the PD-1 checkpoint inhibitor inhibits the binding of PD-1 to PD-L1 and/or PD-L2.

In some embodiments, PD-1 checkpoint inhibitors include anti-PD-1 antibodies, antigen binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In some embodiments, a PD-1 checkpoint inhibitors reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 checkpoint inhibitor is an anti-PD-1 antibody.

In some embodiments, the PD1 checkpoint inhibitor comprises one or more anti-PD-1 antibodies, including nivolumab and pembrolizumab.

In some embodiments, a PD-1 checkpoint inhibitor is nivolumab described herein (also known as MDX-1 106-04, MDX-1106, ONO-4538, BMS-936558, and OPDIVO® (Bristol-Myers Squibb Co., New York, N.Y.). In some embodiments, the PD-1 checkpoint inhibitor comprises nivolumab, which is at a fixed dose of about 3 mg/kg of the patient's body weight.

In some embodiments, a PD-1 checkpoint inhibitor is pembrolizumab described herein (also known as MK-3475, Merck 3475, KEYTRUDA® (Merck Sharp & Dohme Corp., Whitehouse Station, N.J.) and SCH-900475).

In some embodiments, a PD-1 checkpoint inhibitor is CT-01 1 described herein (also known as hBAT or hBAT-1).

In some embodiments, a PD-1 checkpoint inhibitor is AMP-224 described herein (also known as B7-DCIg).

Also provided is a method for treating cancer, comprising administering to a patient in need thereof a therapeutically effective amount of an immunomodulatory agent and a therapeutically effective amount of a PD-L1 checkpoint inhibitor.

In some embodiments, the PD-L1 checkpoint inhibitor is administered substantially at the same time as the immunomodulatory agent. In some embodiments, the PD-L1 checkpoint inhibitor is administered prior to administration of the immunomodulatory agent. In some embodiments, PD-L1 checkpoint inhibitor is administered after administration of the immunomodulatory agent to the patient.

PD-L1 checkpoint inhibitors include but are not limited to molecules that decrease, block, inhibit, abrogate or interfere with signal transduction resulting from the interaction of PD-L1 with either one or more of its binding partners, such as PD-1 and B7-1. In some embodiments, a PD-L1 checkpoint inhibitor is a molecule that inhibits the binding of PD-L1 to its binding partners. In some embodiments, the PD-L1 checkpoint inhibitor inhibits binding of PD-L1 to PD-1 and/or B7-1.

In some embodiments, the PD-L1 checkpoint inhibitor includes one or more anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 or B7-1. In some embodiments, a PD-L1 checkpoint inhibitor reduces the negative co-stimulatory signal mediated by or through cell surface proteins expressed on T lymphocyte-mediated signaling through PD-L1 so as to render a dysfunctional T-cell less dysfunctional (e.g., enhancing effector responses to antigen recognition).

In some embodiments, an anti-PD-L1 antibody is YW243.55.S70.

In some embodiments, an anti-PD-L1 antibody is MDX-1 105 (also known as BMS-936559).

In some embodiments, an anti-PD-L1 antibody is MPDL3280A.

In some embodiments, an anti-PD-L1 antibody is MEDI4736.

The PD-1 or PD-L1 checkpoint inhibitors may be present in a composition and/or administered to a patient in a therapeutically effective amount and, in some embodiments, in a therapeutically effective amount that produces a synergy when the inhibitor is administered together with the immunomodulatory agent. Such an effective amount may vary according to patient characteristics, including gender, size, age, cancer type, cancer stage, route of administration, patient tolerance, toxicity or side effects, and other factors that a skilled medical practitioner would take into account when establishing appropriate patient dosing.

In some embodiments, the PD-1 or PD-L1 checkpoint inhibitors are administered to a patient in an amount of from about 0.5 mg/kg of patient body weight to about 5 mg/kg of patient body weight. The PD-1 or PD-L1 checkpoint inhibitors may be administered to a patient in an amount of from about 1 mg/kg of patient body weight to about 5 mg/kg of patient body weight, from about 1 mg/kg of patient body weight to about 4 mg/kg of patient body weight, from about 1 mg/kg of patient body weight to about 3 mg/kg of patient body weight, from about 2 mg/kg of patient body weight to about 5 mg/kg of patient body weight, from about 2 mg/kg of patient body weight to about 4 mg/kg of patient body weight, from about 3 mg/kg of patient body weight to about 5 mg/kg of patient body weight, or from about 3 mg/kg of patient body weight to about 4 mg/kg of patient body weight. The PD-1 or PD-L1 checkpoint inhibitors may be administered to a patient in an amount of about 1 mg/kg of patient body weight, about 2 mg/kg of body weight, about 3 mg/kg of body weight, about 4 mg/kg of body weight, or about 5 mg/kg of patient body weight. Lesser or greater amounts of the PD-1 or PD-L1 checkpoint inhibitors may be administered.

Also provided is a method for treating cancer, comprising administering to a patient in need thereof a therapeutically effective amount of an immunomodulatory agent, a therapeutically effective amount of a PD-1 checkpoint inhibitor, and a therapeutically effective amount of a PD-L1 checkpoint inhibitor.

In some embodiments, the immunomodulatory agent comprises IFN-γ. In some embodiments, the immunomodulatory agent comprises IFN-γ 1b. In some embodiments, the IFN-γ is human IFN-γ. In some embodiments, the IFN-γ 1b is human IFN-γ 1b. In some embodiments, the IFN-γ 1b consists of between 130 and 146 amino acids. In some embodiments, the IFN-γ l1b consists of between 140 and 146 amino acids. In some embodiments, the IFN-γ 1b is recombinant IFN-γ 1b. An exemplary amino acid sequence for recombinant IFN-γ is as follows:

CYCQDPYVKEAENLKKYFNAGHSDVADNGTLFLGILKNWKEESDRKI
MQSQIVSFYFKLFKNFKDDQSIQKSVETIKEDMNVKFFNSNKKKRDD
FEKLTNYSVTDLNVQRKAIHELIQVMAELSPAAKTGKRKRSQMLFRG
RRASQ

The dose of IFN-γ 1b for treating the cancers may vary depending upon the manner of administration, age, the body weight of the subject, and the condition of the subject to be treated, among other factor. The dose of IFN-γ 1b administered to a patient should be sufficient to effect a beneficial response in the subject. The dose will be determined by the efficacy of the IFN-γ 1b combined with a PD-1/PD-L1 inhibitor and the condition of the subject, as well as the body weight (measured in mass by kg, or in size by m²) of the patient and other conditions and factors as described above. The size of the dose also will be determined by the existence, nature, and extent of any adverse side-effects that accompany the administration of IFN-γ 1b combined with a PD-1/PD-L1 inhibitor in a particular subject. Administration of IFN-γ 1b can be accomplished via single or divided doses.

In some embodiments, the IFN-γ 1b is administered to a patient in an amount of from about 10 μg/m² to about 150 μg/m². The IFN-γ 1b may be administered to a patient in an amount of from about 20 μg/m² to about 120 μg/m², from about 25 μg/m² to about 110 μg/m², from about 30 μg/m² to about 100 μg/m², from about 30 μg/m² to about 75 μg/m², from about 30 μg/m² to about 60 μg/m², from about 30 μg/m² to about 50 μg/m², from about 40 μg/m² to about 80 μg/m², from about 40 μg/m² to about 60 μg/m², from about 50 μg/m² to about 90 μg/m², from about 50 μg/m² to about 80 μg/m², from about 60 μg/m² to about 80 μg/m², from about 70 μg/m² to about 80 μg/m², or from about 80 μg/m² to about 100 μg/m². In some embodiments, the interferon-gamma 1b (IFN-γ 1b) is at a dose of about 30 mcg/m². In some embodiments, the interferon-gamma 1b (IFN-γ 1b) is at a dose of about 50 mcg/m². In some embodiments, the interferon-gamma 1b (IFN-γ 1b) is at a dose of about 75 mcg/m². In some embodiments, the interferon-gamma 1b (IFN-γ 1b) is at a dose of about 100 mcg/m².

Formulations of IFN-γ 1b suitable for administration may include pharmaceutically acceptable excipients, including but not limited to, aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In some embodiments, the IFN-γ 1b is formulated in micelles or liposomes.

In some embodiments, the cancer is chosen from solid tumors.

In some embodiments, the cancer has progressed despite treatment of the patient with at least one prior systemic therapy, which may include prior immunotherapy.

In some embodiments, the cancer is chosen from genitourinary cancers, including urothelial carcinoma and renal cell carcinoma.

In some embodiments, the cancer is chosen from advanced stage tumors.

In some embodiments, the cancer is chosen from metastatic tumors.

In some embodiments, the cancer is chosen from breast, kidney, esophagus, and ovary.

In some embodiments, the cancer is chosen from melanoma, non-small cell lung cancer, blood cancer (e.g., B cell lymphoma, Hodgkin's lymphoma, multiple myeloma), brain cancer (e.g., glioblastoma, glioma, meningioma), bladder cancer, cervical cancer, colorectal cancer, microsatellite instability-high (MSI-H) metastatic colorectal cancer (mCRC), endometrial adenocarcinoma, gastrointestinal cancer, hepatocellular carcinoma, Merkel cell carcinoma, mesothelioma, pancreatic cancer, prostate cancer, small cell lung cancer, and squamous cell cancer of the head and neck (SCCHN)

In some embodiments, the cancer has metastasized into the lymph nodes, liver, bones, pancreas, lungs, kidney, pleura, pericardium, and/or peritoneum.

In some embodiments, the patients are human.

Also provided are compositions that combine one or more PD-1 checkpoint inhibitors with an immunomodulatory agent, as well as compositions that combine one or more PD-L1 checkpoint inhibitors with an immunomodulatory agent, as well as composition that combine one or more inhibitors of each type with the immunomodulatory agent. The compositions may include a carrier, including a pharmaceutically acceptable carrier.

In some embodiments, the pharmaceutical composition comprises a therapeutically effective amount of an immunomodulatory agent in combination with a therapeutically effective amount of a PD-1 checkpoint inhibitor.

In some embodiments, the pharmaceutical composition comprises a therapeutically effective amount of an immunomodulatory agent in combination with a therapeutically effective amount of a PD-L1 checkpoint inhibitor.

In some embodiments, the pharmaceutical composition comprises: a therapeutically effective amount of an immunomodulatory agent; a therapeutically effective amount of a PD-L1 checkpoint inhibitor; and a therapeutically effective amount of a PD-1 checkpoint inhibitor.

A pharmaceutical composition may be in liquid form, or in a dry or lyophilized form that may be reconstituted just prior to administration. In one embodiment, a pharmaceutical composition comprises a lyophilized formulation comprising an effective amount of IFN-γ or IFN-γ 1b and a buffer that maintains the pH of the composition, when reconstituted with water for injection, within the range of 4.0 to 6.0. In one embodiment, a pharmaceutical composition comprises a lyophilized formulation comprising an effective amount of IFN-γ or IFN-γ 1b and a buffer that maintains the pH of the composition, when reconstituted with a sterile saline solution, within the range of 4.0 to 6.0. In one embodiment, a pharmaceutical composition comprises a lyophilized formulation comprising an effective amount of IFN-γ or IFN-γ 1b and a buffer that maintains the pH of the composition, when reconstituted with sterile saline containing between 0.45% (w/v) and 0.9% (w/v) of sodium chloride, within the range of 4.0 to 6.0. The lyophilized formulations can be reconstituted with water for injection and are suitable for use as an injectable for the treatment methodologies described or exemplified herein. In some embodiments, IFN-γ 1b leads to increased PD-L1 expression by tumor cells and thus results in an improved ORR with PD-1 inhibition. In some embodiments, combined immunotherapy with a PD-1/PD-L1 inhibitor and IFN-γ 1b in patients with advanced solid tumors who have demonstrated progression of disease on at least one prior systemic therapy in the metastatic setting leads to ORR.

### EXAMPLES

The following examples are presented only by way of illustration and to assist one of ordinary skill in using the invention. The examples are not intended in any way to limit the scope of the invention.

### Example 1

A phase I study evaluating a combination immunotherapy regimen involving an induction phase of IFN-γ 1b followed by combined treatment with IFN-γ 1b and the PD-1 inhibitor nivolumab was conducted for patients with select advanced solid tumors that have progressed after at least one systemic therapy.

Certain objectives of the study are: to evaluate the investigator assessed overall RR using standard response evaluation criteria in solid tumors (RECIST) version 1.1 for each expansion cohort separately; to evaluate median progression free survival (PFS) for each expansion cohort separately; to evaluate median overall survival (OS) for each expansion cohort separately; to assess OS at 1 year for patients in the expansion cohorts; and to investigate the relationship between PD-L1 expression on tumor cells and on immune cells in the tumor microenvironment before and after treatment initiation

Other objectives of the study are: to investigate whether the change in PD-L1 expression in tumor biopsy samples correlates with ORR; to assess the effect of IFN-gamma treatment on markers of IFN-gamma activity at various time points before, during, and after drug administration; to evaluate for changes in soluble PD-L1 concentrations and PD-1 expression on circulating immune cells before and during study treatment; and to explore the utility of the PanCancer Immune Profiling Panel generated with the Nanostring nCounter® Analysis System platform to provide information regarding immune profiles in the tumor microenvironment from tumor biopsy specimens that supports established immunohistochemical and cytometric results.

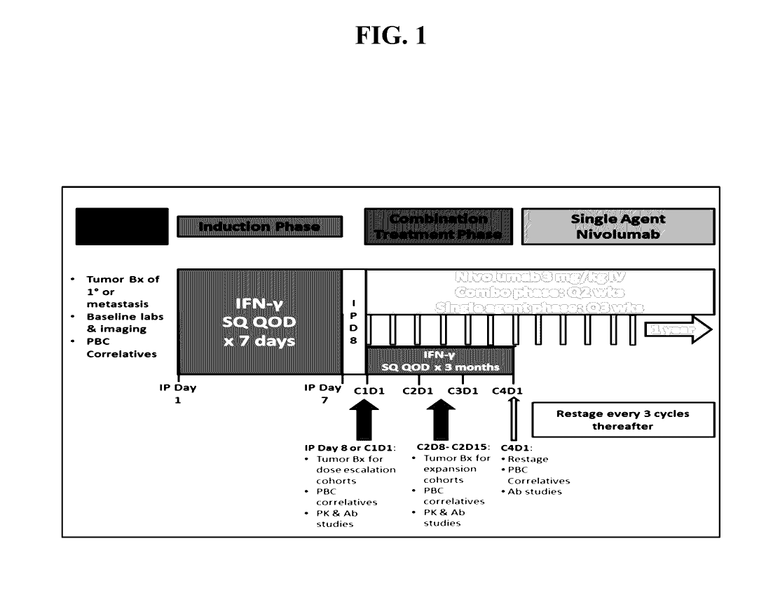

Eligible patients must have a tumor with publicly available evidence of sensitivity to PD-1 pathway inhibition. Treatment with prior PD-1 inhibitors is allowed. Patients will receive IFN-gamma for a one-week period as induction, followed by combination therapy for three months, concluding with single agent nivolumab for up to one year. The study schema is outlined in FIG. 1.

Inclusion criteria are as follows:
Confirmed diagnosis of any metastatic solid tumor will be allowed for initial dose finding cohorts.
Expansion phase will include cohorts consisting of patients with either metastatic urothelial carcinoma (UC) or metastatic renal cell carcinoma (RCC).
Received at least one line of systemic therapy in the metastatic setting. Prior immunotherapy is allowed.
Must have measurable disease that is amenable to biopsy on at least 2 occasions.
Eastern Cooperative Oncology Group (ECOG) performance status of 0 or 1.

Exclusion criteria are as follows:
Diagnosis of immunosuppression or receiving systemic steroid therapy or any other form of immunosuppressive therapy within 7 days prior to the first dose of study treatment.
Not recovered from adverse events due to agents administered more than 4 weeks earlier.
Washout period from any previous chemotherapy, targeted small molecule therapy or radiation therapy within 2 weeks prior to study Day-7 or not recovered from adverse events due to a previously administered agent.
Known active central nervous system (CNS) metastases and/or carcinomatousmeningitis. Asymptomatic, treated, and/or stable brain metastases as measured by subsequent radiologic evaluations at least 3 months apart are permitted.
A history of any autoimmune disease requiring systemic treatment at any time, or any syndrome that requires systemic steroids or immunosuppressive agents.

Nivolumab will be administered as a fixed dose of 3.0 mg/kg intravenously (IV) over one hour on an every two week schedule during the combination phase with IFN-γ 1b, but will be adjusted to every three weeks during the single agent phase. The every two-week dosing schedule during the combination phase follows the FDA-approval for this agent in melanoma and NSCLC and will allow for shorter period of combination therapy. The change to every three week dosing is based on a randomized phase II trial recently published by Motzer and colleagues evaluating the dose-response relationship of varying doses of nivolumab given every three weeks in patients with metastatic renal cell carcinoma (RCC). The results suggested no dose-response relationship existed, though there was a non-significant trend towards greater toxicity at higher doses.

IFN-γ 1b will be self-administered as a subcutaneous (SQ) injection at a starting dose of 50 mcg/m² on an every other day basis. This starting dose was selected based on data from clinical data evaluating the immunologic effects and tolerability of various dose levels, schedules, and routes of IFN-γ 1b administration. This phase I dose-finding study of patients with resected melanomas demonstrated evidence of enhanced immunologic activity (as determined by repeated measurements of hydrogen peroxide levels from monocytes and natural killer cell activity) at various doses and routes of administration. Based on their results and conclusions, IFN-γ 1b dosing from 10-100 mcg/m² achieved consistently high immunologic effects with a tolerable side effect profile. Their data also suggested that administration SQ every other day may be the optimal dose to maintain immunologic pressure. IFN-γ 1b FDA-approved dosing for CDG and SMO is 50 mcg/m², SQ, three time a week.

The study incorporates a dose-escalation design as outlined in Table 1 with six patients enrolled in each of the three dose levels of IFN-gamma, maintaining a constant nivolumab dose, to determine a recommended phase 2 dose (RP2D) combination.

TABLE 1
IFN-γ 1b Dose Levels for Dose Finding during Induction Phase

| Dose Level | Dose (every other day) |
|---|---|
| −2 | 15 mcg/m² |
| −1 | 30 mcg/m² |
| 1 | 50 mcg/m²* |
| 2 | 75 mcg/m² |
| 3 | 100 mcg/m² |

*Bold typed denotes starting dose.

Dose limiting toxicities (DLT) are assessed during the first 6 weeks of combination therapy and the decision rules are outlined in Table 2. Once a RP2D is established, two separate 15 patient expansion cohorts in renal cell carcinoma and urothelial carcinoma will be enrolled to evaluate the combination for safety and preliminary efficacy in two homogenous populations. The primary endpoint is safety, with secondary endpoints including ORR, PFS, OS, and landmark survival. Patients must also undergo pre- and on-treatment tumor biopsies to assess PD-L1 expression changes. Correlative peripheral blood analyses are also embedded in the study.

TABLE 2
GU-084 Decision Rules for Dose Escalation and Cohort Expansion

| Number of patients with | |
|---|---|
| DLT | Escalation and expansion decision rules |
| 0-1 out of 6 | If first or second dose level evaluated, escalate |
| | to next highest dose |
| | If third dose level evaluated (or more), declare |
| | this dose the PP2D and proceed to opening |
| | expansion cohorts |
| ≧2 out of 6 | Consider this dose unacceptable; de-escalate |
| | dose level and open new expansion cohort of |
| | 6 patients at next lowest dose level |
| | If third cohort (dose level −2), terminate study |

IFN-γ 1b will be self-administered as a SQ injection at an initial dose of 50 mcg/m² on an every other day basis (dose level 1). A one week induction phase of IFN-γ 1b alone will allow for assessment and management of any IFN-related toxicity, as well as provide a potential window for “immunologic priming” as PD-L1 is up-regulated. This period will be counted from day-7 (the first IFN-γ 1b injection) through day-1.

Using a standard 3+3 design, the initial cohort of patients enrolled at dose level 1 also will be monitored for evidence of an immunologic response to IFN-γ 1b as assessed by peripheral blood cell (PBC) markers of response to IFN-γ 1b (STAT-1, STAT-1p, and MHC Class I expression) and PD-L1 expression on tumor biopsy. If no DLTs are recorded and both the PBC markers and PD-L1 expression on tumor biopsy indicate effect of the IFN-γ 1b, 50 mcg/m² will be the dose used for the dose expansion phase. If one or more patients experiences a DLT or does not achieve an adequate immunologic response, three further patients will be accrued at that dose level. If no DLTs are reported and the immunologic response is inadequate in at least 2/3 patients, the next cohort will accrue at a higher dose level. Nivolumab dosing will remain fixed at 0.3 mg/kg.

The induction phase of IFN-γ 1b will begin on day-8 and proceed with every other day administration though day-1. There will be a day off from treatment (pursuant to the alternative day dosing of IFN-γ 1b) and there will be an appropriate day to schedule on-treatment biopsy and blood collection for standard of care (SOC) and correlative analyses for the first cohorts of patients. Cycle 1 day 1 (C1D1) will denote the start of the combined therapy phase of the study, on which day patients will receive IFN-γ 1b as per their induction dose level and start treatment with a fixed dose of nivolumab at 3.0 mg/kg. Biopsy and peripheral blood draw for SOC and correlative analyses may also be procured on this day.

During the combined therapy phase, nivolumab will be administered at the fixed dose of 3.0 mg/kg as an intravenous (IV) infusion over 60 minutes every 2 weeks. A cycle will be defined as every 28 days, thus two doses of nivolumab will be given during each cycle on days 1 and day 15. IFN-γ 1b administration will continue on an every other day schedule as per the induction phase dosing for each patient cohort. The drugs will be administered until disease progression, intolerable toxicity, or study withdrawal for any reason.

IFN-γ 1b, as combination therapy, will be continued on an every other day basis for a duration of 3 months, at which point patients who are clinically benefitting will stop IFN-γ 1b but continue treatment with nivolumab for up to 2 years if they continue to exhibit a beneficial response. Once patients proceed to single agent treatment with nivolumab, the schedule will change to administration every three weeks, as outlined above.

As outlined above, dose expansion will commence once a safe, tolerable, and immunologically active dose of IFN-γ 1b has been established. Dose expansion will be planned only in patients with metastatic urothelial carcinoma and renal cell carcinoma who otherwise fit the inclusion and exclusion criteria outlined above.

During the combined treatment phase of dose expansion, intra-patient dose reductions of IFN-γ 1b will be permitted. Nivolumab dosing will remain fixed.

A dose delay of up to three weeks will be allowed for adverse events (AEs) to allow for recovery to grade 1 AEs as defined by the Common Terminology Criteria for Adverse Events (CTCAE).

Patients in the dose expansion cohorts will not be required to undergo biopsy on day 0 or C1D1. Peripheral blood for SOC and correlative analyses will still be required on these days. However, a biopsy will be required for these patients instead on or about C2D8 (after three doses but before four doses of nivolumab). All patients will still undergo a biopsy at baseline prior to starting the induction phase.

The target sample size will be include 6-21 patients with solid tumors in the phase I, and 15 patients each in the RCC and UC expansion cohorts for a total of between 6 and 54 patients

Consented patients will undergo baseline imaging with computed tomography (CT) of the chest, abdomen, and pelvis and an optional nuclear medicine bone scan if suspected or known bony metastatic disease.

Baseline laboratory studies including a complete blood count (CBC), a complete metabolic panel (CMP) and thyroid stimulating hormone (TSH) will be assessed prior to starting induction therapy with IFN-γ 1b on Day-8. CBC and CMP will be reassessed on C1D1, then every two weeks thereafter. TSH will be assessed again four weeks after the first dose of nivolumab and every 28 days thereafter.

Baseline biopsy of primary or metastatic site will be performed within seven days prior to initiation of treatment with IFN-γ 1b. For patients in the dose finding cohort, a second biopsy of the same site will be performed at the conclusion of the induction phase but prior to initiation of the first dose of nivolumab (either day 0 or C1D1). Once the study moves to the dose expansion cohort, the second biopsy will be performed during combination therapy after the third dose of nivolumab but prior to the fourth dose (optimally C2D8). Detail of analysis of biopsy specimens can be reviewed below.

Correlative studies via peripheral blood draws (see further detail below) will be collected with initial lab studies prior to day-8, then again prior to administration of the first dose of nivolumab (day 0 or C1D1), on C2D8, and at the completion of combination therapy (C4D1).

Patients will be evaluated with a history and physical exam (H&P) on Day-7 of treatment prior to starting IFN-γ 1b. Patient or designated surrogate will receive teaching on how to administer the SQ injection and will be witnessed injecting first dose. Patient will then be seen on C1D1 for H&P prior to administration of the first dose of nivolumab. They will then be evaluated with an H&P every two weeks for the first 3 months, then every six weeks thereafter.

Patients will be assessed for any AEs during treatment that could be related to the study drugs. ABs will be recorded and graded by CTCAE 4.0.

Specifically, patients will be monitored closely for immune-related adverse events (irAEs) that have been previously reported with checkpoint inhibitors. Management decisions of any irAEs will be made by the treating physician using previously established recommendations as guidance.

Tumor assessments will be performed at baseline with CT of the chest, abdomen, and pelvis and optional bone scan as above. Restaging will then occur after 6 weeks of combination therapy (C2D14) and at the conclusion of the combination phase (C4D1). For patients who remain on single agent nivolumab, subsequent restaging scans will occur every 3 months thereafter. For patients with known or presumed metastatic disease to the bone, nuclear medicine bone scans will be performed with every CT scan.

Determination of progressive disease will be defined +by RECIST v1.1 and patients who are deemed to be clinically benefitting by the treating physician can be treated beyond first radiographic progression.

### Correlative Studies.

I. Biopsy Specimen Analysis
   A. Biopsies will be performed at 2 time periods for each patient
      1. All patients will undergo a baseline biopsy within seven days of initiation of induction treatment with IFN-γ 1b.
      2. Patients in the initial dose finding cohort will undergo a second biopsy at the conclusion of the induction phase but prior to starting treatment with nivolumab (either day 0 or C1D1).
      3. For patients in the dose expansion cohort, the second biopsy will be performed during the combination therapy phase after the third dose of nivolumab but prior to the fourth dose (optimally C2D8).
      4. Analyses to be performed on biopsy samples:
         a. Immunohistochemistry (IHC) to assess expression of PD-L1, MHC class I, and phosphoSTAT-1 in the tumor specimen. Other IHC analyses may include but are not limited to: CD3, CD4, CD8. Intensity of staining will be graded and compared between baseline and on-treatment sample.
         b. Nanostring nCounter® (NanoString Technologies, Inc., Seattle, Wash.) Analysis System
            i. New technology expected to soon be available at Fox Chase Cancer Center that uses molecular “barcodes” attached to a single target-specific probe corresponding to a gene of interest to yield analyses of gene expression comparable to quantitative polymerase chain reaction (qPCR) but without a need for amplification of target molecules. This technology can be used for DNA or RNA in fresh/frozen or formalin fixed paraffin embedded (FFPE) tissue.
            ii. Recently released PanCancer Immune Profiling Panel is a 770 gene panel that enables assessment of most markers of immune expression in a tumor sample, including cytokines, chemokines, tumor infiltrating lymphocytes (TILs) and immune checkpoint genes.
            iii. While a relatively new technology, the hope is that this will provide infinitely more information from one tissue sample with much less tissue and may ultimately replace standard, subjective pathology techniques.
            iv. Correlation of the results from this technology compared to more standard assessments of the outcomes we are interested in (markers of IFN-γ 1b activity, presence of TILs, PD-L1 expression) may enable less invasive modes of tissue acquisition and improved clinicopathologic precision in the future.
II. Peripheral Blood Analysis
   A. Peripheral blood will be collected to evaluate for correlative analyses at four time points (baseline, day 0/C1D1, C2D8, C4D1).
   B. Analyses to be performed on peripheral blood: IFN-γ 1b immunogenicity: IFN-γ 1b administration reliably leads to up-regulation of STAT1, STAT-1p, and MHC class I. For the patients in the dose finding cohort, measurements of the change in these levels from baseline to biopsy after IFN-γ 1b induction will be analyzed. When assessed in conjunction with the change in PD-L1 expression observed in the biopsy specimens, pre-specified criteria will determine whether the IFN-γ 1b dose is resulting in the expected and desired immunogenic effect. Additionally, as noted in a prior trial of biomarkers in patients treated with nivolumab, CXCL9/10 have been used as surrogates for increased IFN-γ 1b activity and can be assessed in serum. The effect of combination treatment on these factors will also be assessed at two time points after the addition of nivolumab.
   C. PD-1/PD-L1 response: Utilizing flow cytometry, we will evaluate the changes of PD-L1 expression on peripheral blood cells including dendritic cells and T-cells. If these correlate with the effect seen in tumor tissue, these may serve as a less invasive biomarker of IFN-γ 1b activity. We will also measure levels of soluble PD-1 as well as the expression of PD-1 on circulating immune cells and the relative change from treatment.
III. Pharmacokinetic (PK) and antibody (including neutralizing antibody) studies of IFN-γ and nivolumab.
   A. PK studies for IFN-γ will be performed on C1D1. Patients will self-administer their dose on or about 8 AM. Serum PK studies will be drawn 6-8 hours later, after which time the first dose of nivolumab will be administered.
   B. PK studies for nivolumab will be conducted during the combined treatment phase and will be collected for all patients on C2D8, time points will be determined.
   C. Antibody studies for IFN-γ will be performed prior to dosing on Day-7 and on C1D1, C4D1 and 2-3 months post combination dosing. Antibody studies for nivolumab will be performed prior to dosing on C1D1, C4D1 and 2-3 months during single agent treatment and 1 month post single agent treatment.

Results from the study were obtained as described in the phase 1 study. Patients enrolled in the study suffered from primary cancer of the kidney, breast, esophagus, and ovary, with metastases to other organs including lymph nodes, liver, bones, pancreas, lungs, pleura, pericardium, and peritoneum. Female patient ages ranged from 32 to 59. Male patient ages ranged from 36 to 57. The great majority of reported adverse events (AEs) causally-related to IFN-gamma and/or nivolumab were grade 1 in severity with a few grade 2 AEs. Most IFN-gamma-related AEs were constitutional or hematological in nature. Serious AEs (SAEs) were reported in about 40% of patients, including: grade 3 dyspnea/grade 3 fatigue/grade 2 weakness in one patient; grade 2 disease progression in one patient; and grade 2 pain/grade 3 fatigue in one patient. All SAEs were assessed by the investigator as the result of disease progression. IFN-gamma dose reduction was performed in one patient but symptoms of fatigue and weakness did not improve and were ultimately judged as related to disease progression. No immune-related AEs were reported. One patient experienced a grade 2 elevation in hepatic transaminases of unclear etiology, but this was resolved without intervention. There were also mild thyroid hormone changes reported, including a patient with thyroid stimulating hormone (TSH) dropping below the lower limit of normal, and another patient requiring an increased dose of levothyroxine for pre-existing hypothyroidism.

The preliminary flow cytometry findings indicate a trend for IFN-gamma effects on peripheral blood cells using activation and immune checkpoint markers. IFN-gamma 50 mcg/m² administered subcutaneously every other day for 7 days showed the following: monocyte activation (using MHC class II or CD16) in some patients (independent of clinical benefit); no upregulation of MHC class I on B cells; increase of PD-L1 expression on monocytes and T cells in some patients (more in patients without clinical benefit); and increase in PD-1 expression on T cells in some patients (more in patients with clinical benefit).

IFN-gamma 50 mcg/m² administered subcutaneously every other day in combination with nivolumab 3 mg/kg intravenously every 2 weeks was well tolerated in an initial cohort of patients with solid tumors. No DLT was observed. No safety concern was identified. No immune-related AEs occurred although a transient grade 2 hepatic transaminase elevation and a few grade 1 thyroid hormone changes were observed. Such immune-related AEs are expected in approximately 10-15% (≧grade 3) of patients based on prior studies using immune checkpoint inhibitors although grade 1-2 events may occur in 30-40% of patients.

This study demonstrates, even at the lowest dose cohort, that combination therapy with IFN-gamma and a checkpoint inhibitor results in clinical benefit to patients suffering from advanced solid tumors. About 40% of the patients in this study attained a radiographically and/or clinically stabilized disease state. This number is expected to be higher in a population of patients with cancers that over express PD-1 rather than PD-L1. It is also expected that a combination of INF-gamma and a PD-L1 inhibitor would be more effective treating patients having cancers that over express PD-L1 rather than PD-1.

Other uses of the disclosed methods will become apparent to those in the art based upon, inter alia, a review of this patent document.

What is claimed is:

1. A method for treating cancer, comprising administering to a patient in need thereof
(a) a therapeutically effective amount of an immunomodulatory agent, and
(b) a therapeutically effective amount of a PD-1 checkpoint inhibitor and/or a therapeutically effective amount of PD-L1 checkpoint inhibitor.

2. The method according to claim 1, wherein the immunomodulatory agent is administered by itself during an induction phase that is prior to the administration of the PD-1 checkpoint inhibitor and/or the PD-L1 checkpoint inhibitor.

3. The method according to claim 1, wherein the PD-1 checkpoint inhibitor comprises an anti-PD-1 antibody.

4. The method according to claim 3, wherein the PD-1 checkpoint inhibitor is selected from nivolumab and pembrolizumab.

5. The method according to claim 1, wherein the PD-L1 checkpoint inhibitor comprises an anti-PD-L1 antibody.

6. The method according to claim 5, wherein the PD-L1 checkpoint inhibitor is selected from YW243.55.S70, MDX-1 105, MPDL3280A, and MEDI4736.

7. The method according to claim 1, comprising administering to a patient in need thereof (a) a therapeutically effective amount of an immunomodulatory agent, (b) a therapeutically effective amount of a PD-1 checkpoint inhibitor, and (c) a therapeutically effective amount of a PD-L1 checkpoint inhibitor.

8. The method according to claim 1, wherein the immunomodulatory agent comprises interferon gamma (IFN-γ) or interferon gamma 1b (IFN-γ 1b).

9. The method according to claim 1, wherein the patient is a human.

10. The method according to claim 1, wherein the cancer is a solid tumor cancer.

11. The method according to claim 1, wherein the cancer is selected from the group consisting of breast cancer, kidney cancer, esophageal cancer, ovarian cancer, urothelial carcinoma, renal cell carcinoma, genitourinary cancer, melanoma, non-small cell lung cancer, blood cancer, B cell lymphoma, Hodgkin's lymphoma, multiple myeloma, brain cancer, glioblastoma, glioma, meningioma, bladder cancer, cervical cancer, colorectal cancer, endometrial adenocarcinoma, gastrointestinal cancer, hepatocellular carcinoma, Merkel cell carcinoma, mesothelioma, microsatellite instability-high metastatic colorectal cancer, pancreatic cancer, prostate cancer, small cell lung cancer, and squamous cell cancer of the head and neck.

12. A pharmaceutical composition, comprising: (a) a therapeutically effective amount of an immunomodulatory agent; and (b) a therapeutically effective amount of a PD-1 checkpoint inhibitor and/or a therapeutically effective amount of a PD-1 checkpoint inhibitor.

13. The pharmaceutical composition according to claim 12, wherein the PD-1 checkpoint inhibitor comprises an anti-PD-1 antibody.

14. The pharmaceutical composition according to claim 13, wherein the anti-PD-1 antibody is selected from nivolumab and pembrolizumab.

15. The pharmaceutical composition according to claim 14, wherein the anti-PD-1 antibody is nivolumab and at a fixed dose of 3.0 mg/kg.

16. The pharmaceutical composition according to claim 14, wherein the PD-L1 checkpoint inhibitor comprises an anti-PD-L1 antibody.

17. The pharmaceutical composition according to claim 16, wherein the anti-PD-L1 antibody is selected from YW243.55.S70, MDX-1 105, MPDL3280A, and MEDI4736.

18. The pharmaceutical composition according to claim 12, wherein the immunomodulatory agent comprises interferon gamma (IFN-γ) or interferon-gamma 1b (IFN-γ 1b).

19. The pharmaceutical composition according to claim 18, wherein the interferon-gamma 1b (IFN-γ 1b) is at a dose of at least about 30 mcg/m², at least about 50 mcg/m², at least about 75 mcg/m², or at least about 100 mcg/m².

20. The pharmaceutical composition according to claim 12, comprising: (a) a therapeutically effective amount of an immunomodulatory agent; (b) a therapeutically effective amount of a PD-1 checkpoint inhibitor, and (c) a therapeutically effective amount of a PD-1 checkpoint inhibitor.
